(19) United States
(12) Patent Application Publication
(10) Pub. No.: US 2006/0003027 A1
(43) Pub. Date: Jan. 5, 2006

(54) Composition and method for reducing side effects of indole-3-carbinol and derivatives

(76) Inventor: James H. Zhou, Middletown, CT (US)

Correspondence Address: BACHMAN & LAPOINTE, P.C., 900 CHAPEL STREET, SUITE 1201, NEW HAVEN, CT, 06510, US

(21) Appl. No.: 10/882,046

(22) Filed: Jun. 30, 2004

(51) Int. Cl. A61K 33/04; A61K 31/714; A61K 31/404; A61K 31/375; A61K 31/59; A61K 31/525

(52) U.S. Cl. 424/702; 514/167; 514/52; 514/251; 514/350; 514/474; 514/415

(57) ABSTRACT

A method is provided for reducing side effects while treating a mammal using indole-3-carbinol, which method includes the steps of administering an effective amount of indole-3-carbinol along with side effect-reducing amounts of a side-effect reducing component. A composition is also provided, as are various combinations of side effect-reducing agents.

### FIELD OF THE INVENTION

This invention relates to reducing side effects of indole-3-carbinol (I3C) and/or derivatives in the treatment and prevention of diseased conditions. This invention provides a method to allow long term use of indole-3-carbinol (I3C) at an effective dosage for preventing and treating these conditions from progressing into cancer.

### BACKGROUND OF THE INVENTION

Numerous naturally occurring compounds exhibit preventative effects against various cancers. They are, however, rather weak in action. A high dosage is often needed, which normally exceeds the amount that one can intake through a regular diet. Such a high dosage may in general cause undesirable side effects. Indole-3-carbinol (I3C), also known as 1H-indole-3-methanol, is one of many naturally occurring compounds found in cruciferous vegetables. Its chemical structure has been well known. It has been used as a dietary supplement to aid in the inhibition of laryngeal papillomatosis, to benefit humans in connection with decreased cancers of the colon, lung, and breast. (Wattenburg, L. W. & Loub, W. D., “Inhibition of Polycyclic Aromatic Hydrocarbon-Induced Neoplasia by Naturally Occurring Indoles,” Vol. 38, Cancer Res., 1410-1413, (1978)). It has also been suggested that a high dosage of I3C may exhibit therapeutic benefits in connection with treatment of cervical or vaginal neoplasia. The present invention includes compositions and methods for reducing side effects of I3C and its derivatives in mammals.

Indole-3-carbinol (I3C) as found in crucifers may be specifically responsible for preventing or halting carcinogenesis in certain cases. (Grubbs C. J. et al., Chemoprevention of Chemically-Induces Mammary Carcinogenesis by Indole-3-Carbinol,” Vol. 15, Anticancer Res., 709-716, (1995)). Grubbs et al. showed I3C was effective in inhibiting mammary carcinogenesis in a rat model. Newfield et al. showed that I3C inhibits growth of HPV-induced benign tumors of laryngeal tissue in a mouse model. (Newfield et al., Estogen Metabolism & Human Papillmavirus-Induced Tumors of the Larynx: Chemo-Prophylaxis with Indole-3-Carbinol,” Vol. 13, Anticancer Res., 337-341, (1993)). Although Newfield et al. suggested the possibility that a diet high in cruciferous vegetables or the consumption of an I3C supplement could perhaps be used to prevent human papillomavirus-induced tumors of the larynx or genital tract, no evidence was presented that I3C would effectively treat cervical interaepithelial neoplasia (CIN) in either mice or humans. (Newfield et al., at 340). Similarly, I3C has been used as a dietary supplement for humans to aid in the inhibition of laryngeal papillomatosis. (Coll, D. et al., “Treatment of recurrent respiratory Papillomatosis with Indole-3-carbinol,: Vol. 18, Am. J. Otolaryngol., 283-285, (1997)).

Recently, Bell et al. claimed evidence that I3C would treat CIN or other forms of cervical or vaginal neoplasia in humans (Bell; Maria, 562 Tolland Drive., Castle Rock, Colo. 80104)

Schmidt-Grimminger; Delf-Christian (562 Tolland Dr., Castle Rock, Colo. 80104). Bell's method, however, describes long term use of I3C in a dosage at 200 mg/day/person and 400 mg/day/person to treat cervical or vaginal neoplasia in humans. It has been found, however, that I3C-causes side effects in the course of trial. We have discovered that “the effective” dosage at 200 mg/day of I3C and above 200 mg/day may cause undesirable side effects including headache, dizziness, stomach disturbance, convulsion, vomiting, and fatigue, and more when given to some people in a regular body weight. The side effects of effective I3C dosage prohibit long term use of this valuable compound in treating or preventing many diseases including neoplasia in the larynx or genital tract, colon, lung and breast.

It was discovered that use of I3C at a high dosage in the range of 200 mg/day to 400 mg/day (Bell et al) is beneficial to cervical or vaginal neoplasia. Use of I3C at a high dosage, however, may cause undesirable side effects including headache, dizziness, upset stomach, vomiting, and more when given to some people. The side effects of effective I3C dosage prohibit long term use of this valuable compound in treating or preventing many diseases. Therefore, there is currently no safe/effective chemo-preventative method for a long term use of I3C in treating or preventing the progression of precancerous tissue to cancer.

It is therefore the primary object of the present invention to provide a composition allowing delivery of effective amounts of I3C to mammals with reduced side effects.

It is a further object of the invention to provide a method for administering I3C with reduced side effects.

Other objects and advantages of the present invention will appear herein below.

### SUMMARY OF THE INVENTION

In accordance with the present invention, the foregoing objects and advantages have been readily attained.

According to the invention, a method is provided for reducing side effects while treating a mammal using indole-3-carbinol, which method comprises the steps of administering an effective amount of indole-3-carbinol along with side effect-reducing amounts of a side-effect reducing component.

Still further according to the invention, an indole-3-carbinol-containing composition is provided which comprises an effective amount of indole-3-carbinol and a side effect-reducing amount of a side-effect reducing component.

### DETAILED DESCRIPTION

The invention relates to a composition including indole-3-carbanol (I3C) with additional agents for reducing side effects of I3C. The invention further relates to a method for administering I3C wherein side effects normally accompanying same are reduced.

It has been found that use of I3C in various applications can be extremely beneficial in treating potentially serious illnesses in mammals including humans. However, as set forth above, in order to obtain suitable results it is frequently necessary to use I3C in amounts which cause undesirable side effects including headache, nausea, dizziness, upset stomach, vomiting and the like. In accordance with the present invention, I3C is administered along with effective amounts of a side effect-reducing component, especially one including vitamin C or ascorbic acid and other advantageous ingredients as will be discussed below, whereby I3C can be used effectively without the normally experienced side effects.

According to the invention, this composition allows for treatment or prevention of many diseases including neoplasia in the larynx or genital tract, colon, lung, breast and the like.

The indole-3-carbinol containing composition can be any I3C composition including derivatives and the like. Examples of particularly acceptable I3C includes dimmer 3,3′-dindolylmethane (DIM), indole [3,2b] carbazole (ICz) and combinations thereof.

According to the invention, I3C is also combined in a composition including a side effect-reducing component whereby the composition can be administered to mammals to treat an I3C appropriate illness without causing the aforesaid undesirable side effects.

According to the invention, it has been found that several vitamins and minerals, especially in combinations discussed below, serve to greatly reduce the side effects normally experienced by a mammal using I3C. Useful side effect-reducing agents include vitamin C, selenium, folic acid, pyridoxine (B6), and cyanocobalamin (B12), and especially combinations of these agents. Vitamin C, or ascorbic acid, has been found to be effective when administered along with I3C, and a composition including combinations of the above agents, and especially all of them, has been found to be effective in suppressing the undesirable side effects of I3C.

Additional side-effect-reducing agents can also be used, especially those selected from the group consisting of vitamins A, D, E or K, thiamin (B1), niacinamide (B3), biotin, pantothenic acid, magnesium, zinc, copper, chromium, lycopene, catechin (for example 80% wt. green tea extract), resveratrol and combinations thereof.

When used to treat mammals for the aforesaid ailments, it is desirable to administer I3C in doses of at least about 100 mg/day/100 lbs., preferably between about 150 and about 1000 mg/day/100 lbs, more preferably between about 200 and about 500 mg/day/100 lbs. The weights referred to herein are body weights of the patient being treated.

In accordance with the invention, it has been found that incorporating I3C into a composition including side-effect-reducing agents allows for use of the I3C in amounts which effectively treat serious diseases such as those referred to above.

In accordance with one preferred aspect of the present invention, a particularly preferred side-effect-reducing component is vitamin C, or ascorbic acid. This component can advantageously be incorporated into the composition in amount of at least about 25 mg/day/100 lbs., more preferably between about 37.5 and about 50,000 mg/day/100 lbs., still more preferably between about 50 and about 25,000 mg/day/100 lbs. and ideally between about 500 and 8,000 mg/day/100 lbs.

In further accordance with the above, it is further preferred that I3C and the side-effect-reducing agent or component be incorporated into the composition at a ratio by weight of side-effect-reducing component to I3C of at least about ¼, preferably at least about 1/1, and more preferably at least about 10/1 and up to a practical maximum of about 50/1.

It is believed that I3C generates a by product which must be disposed of by the liver. When administered in high dosage, I3C generates more by product than the liver can normally dispose of, and build up of this by product is believed to cause the undesirable side effects discussed above.

In accordance with the present invention, the side-effect-reducing agent enhances function of the liver in disposing of the by product so as to dispose of the agent which causes the undesirable side effects. Vitamin C is particularly effective at enhancing the function of the liver in this manner. Other suitable components for performing this function include polysaccharides, glycosides, flavanoids, flavones, antioxidants, polyphenols, amino acids and alkaloids, and the like.

As set forth above, vitamin C is a particularly preferred side-effect-reducing component. In addition, it may be desirable to incorporate other components into the composition of the present invention to further enhance various functionality of the combination.

One additional component which is desirable to incorporate into the composition of the present invention is folic acid. Folic acid helps to block side effects by enhancing function of the liver, and further supplements the activity of the I3C component. In accordance with the present invention, folic acid can advantageously be incorporated into the composition in an amount of at least about 50 mcg/day/100 lbs., more preferably between about 100 and 2,000 mcg/day/100 lbs., and most preferably about 800 mcg/day/100 lbs.

Another ingredient which has been found to be useful in accordance with the present invention is selenium. Some cancers are associated with viral activity, and selenium can provide anti-viral activity. Thus, selenium is a useful supplement to the I3C component. Furthermore, selenium can assist in reduction of the side effects experienced in connection with high doses of I3C. Selenium can advantageously be incorporated into the composition in an amount of at least about 10 mcg/day/100 lbs. preferably between about 50 and 700 mcg/day/100 lbs., and ideally in amount of about 70 mcg/day/100 lbs.

A further component which can be desirable to incorporate into the present invention is B6 and/or B12, preferably a combination of same. These components enhance function of the vascular system, and therefore help to deliver I3C and thereby enhance treatment using same. While either of these components separately enhances the vascular system, in combination they have been found to provide the best results. Thus, in accordance with the present invention, the composition can advantageously include both B6 and B12. B6 is preferably present in an amount of at least about 0.5 mg/day/100 lbs., more preferably between about 1 and about 200 mg/day/100 lbs. and most preferably about 10 mg/day/100 lbs. B12 is preferably present in amount of at least about 2 mcg/day/100 lbs., preferably between about 5 and about 100 mcg/day/100 lbs. and ideally about 10 mcg/day/100 lbs.

A preferred composition in accordance with the invention includes each of the above ingredients or components, and can have amounts of these various components preferably as forth in Table 1 below:

TABLE 1

| | | |
|---|---|---|
| Indole-3-carbinol (or derivatives): | 200-500 | mg/day/100 lbs. |
| Vitamin C (Ascorbic acid): | 250-8,000 | mg/day/100 lbs. |
| Folic acid: | 400-2,000 | mcg/day/100 lbs |
| Selenium: | 70-700 | mcg/day/100 lbs |
| B6: | 2-10 | mg/day/100 lbs. |
| B12: | 6-10 | mcg/day/100 lbs. |

As set forth above, one particularly suitable composition is a combination of I3C or its derivatives and vitamin C (ascorbic acid) or its derivatives, and a preferred composition includes in addition to the I3C and vitamin C, a folic acid component, selenium component, B6 and B12. In further accordance with a preferred embodiment of the present invention, the composition can include the ingredients set forth above and, in addition, can include one or more of vitamin A, vitamin D, vitamin E, vitamin K, thiamin (B1), niacinamide/niacin (B3), pyridoxide (B6), biotin, pantothenic acid, magnesium, zinc, copper, chromium, lycopene, catechin (80% green tea extract) and resveratrol.

A particularly desirable composition in accordance with the invention can have each of these components, and can contain them in amounts as forth in Table 2 below:

TABLE 2

| | | |
|---|---|---|
| Indole-3-carbinol (or derivatives): | 200-500 | mg/day/100 lbs. |
| Vitamin C (Ascorbic acid): | 250-8,000 | mg/day/100 lbs. |
| Folic acid: | 400-2,000 | mcg/day/100 lbs. |
| Selenium: | 70-700 | mcg/day/100 lbs. |
| B6: | 2-40 | mg/day/100 lbs |
| B12 | 6-40 | mcg/day/100 lbs. |
| Vitamin A | 1,000-10,000 | I.U./day/100 lbs. |
| Vitamin D | 100-2,000 | I.U./day/100 lbs. |
| Vitamin E | 50-800 | I.U./day/100 lbs. |
| Vitamin K | 10-400 | mcg/day/100 lbs. |
| Thiamin (B1) | 1.5-7.5 | mg/day/100 lbs. |
| Niacinamide (B3): | 10-100 | mg/day/100 lbs |
| Pyridoxine (B6) | 1-30 | mg/day/100 lbs. |
| Biotin | 100-1,000 | mcg/day/100 lbs. |
| Pantothenic Acid | 1-100 | mg/day/100 lbs. |
| Magnesium | 100-500 | mg/day/100 lbs. |
| Zinc | 5-100 | mg/day/100 lbs. |
| Copper | 1-30 | mg/day/100 lbs. |
| Chromium | 10-500 | mcg/day/100 lbs |
| Lycopene | 5-500 | mg/day/100 lbs. |
| Catechin (80% green tea extract) | 5-1,000 | mg/day/100 lbs. |
| Resveratrol | 10-1,000 | mg/day/100 lbs. |

As set forth above, the compositions according to the present invention advantageously allow for use of I3C in effective amounts with greatly reduced if not completely eliminated side effects. This greatly increases the usefulness of I3C in treating various ailments, all within the scope of the present invention.

The following examples further demonstrate the advantageous compositions in accordance with the present invention.

### EXAMPLE 1

A female patient, 33 years old and 110 lbs. in weight, has complained of pain in the area of the coccygeal vertebra, which may reflect cervical inflammation, and/or cervical or vaginal neoplasia. Pap smear tests showed abnormal cells.

This patient was administered a high dosage of I3C, specifically 110 mg/per dose, twice daily. The patient experienced headache, dizziness and fatigue after following this regimen for about a week. The patient did notice relief from the pain in the area of the coccygeal vertebra. A composition (composition 1) in accordance with the present invention was then given to this patient. Composition 1 is as set forth below:

Composition 1
Indole-3-Carbinol (or derivatives): 200 mg/day/100 lbs.
Vitamin C: 3,000 mg/day/100 lbs.

The patient reported no side effects such as headache, dizziness or fatigue while taking composition 1, and after 14 weeks of treatment with composition 1, a pap smear showed negative results.

It is clear that the composition in accordance with the present invention advantageously allows for reduction in side effects in connection with the set forth dosage of I3C.

### EXAMPLE 2

A 34 year old female patient, 116 lbs in weight, had a complaint similar to the patient in example 1, specifically pain in the area of coccygeal vertebra. A pap smear test showed abnormal cells, and further biopsy and microscopic examination revealed inflamed lesions and non-cancerous abnormal cells. Composition 2 in accordance with the present invention was administered to the patient. Composition 2 is as set forth below:

Composition 2:
Indole-3-carbinol (or derivatives): 250 mg/day/100 lbs.
Vitamin C (Ascorbic acid): 250 mg/day/100 lbs.
Folic acid: 800 mcg/day/100 lbs
Selenium: 70 mcg/day/100 lbs
B6: 2 mg/day/100 lbs.
B12: 6 mcg/day/100 lbs.

After about three days of treatment with Composition 2, the pain was relieved. Further, there were no complaints of headache, dizziness and fatigue. Composition 3 was then administered to this patient for a period of eight weeks. Composition 3 is as set forth below:

Composition 3
Indole-3-carbinol (or derivatives): 250 mg/day/100 lbs.
Vitamin C (Ascorbic acid): 250 mg/day/100 lbs.
Folic acid: 800 mcg/day/100 lbs.
Selenium: 70 mcg/day/100 lbs.
B6: 2 mg/day/100 lbs
B12 6mcg/day/100 lbs.
Vitamin A 5,000 I.U./day/100 lbs.
Vitamin D 400 I.U./day/100 lbs.
Vitamin E 60 I.U./day/100 lbs.
Vitamin K 75 mcg/day/100 lbs.
Thiamin (B1) 1.5 mg/day/100 lbs.
Niacinamide (B3): 20 mg/day/100 lbs
Pyridoxine (B6) 2 mg/day/100 lbs.
Biotin 300 mcg/day/100 lbs.
Pantothenic Acid 10 mg/day/100 lbs.
Magnesium 100 mg/day/100 lbs.
Zinc 22.5 mg/day/100 lbs.
Copper 3 mg/day/100 lbs.
Chromium 120 mcg/day/100 lbs
Lycopene 5 mg/day/100 lbs.
Catechin (80% green tea extract) 25 mg/day/100 lbs.
Resveratrol 10 mg/day/100 lbs.

During this treatment, this patient experienced no headache, dizziness nor fatigue. Further, upon conclusion, pap-smear testing showed negative, and further biopsy and microscopic testing showed a 90% improvement in connection with inflamed tissue, and a reduction of abnormal cells of about 70% as compared to the starting point.

Clearly the composition in accordance with the present invention is quite effective in treating the reported conditions while suppressing the formerly experienced side effects normally accompanying I3C.

In the claims to follow, reference is made to Indole-3-carbinol, Vitamin C and various other components. By reference to these components, it is specifically intended to include derivatives of and to the mentioned component. For example, the term Vitamin C includes compositions which form Vitamin C, compositions which contain the functional element of Vitamin C when processed by the patient, and the like.

It is to be understood that the invention is not limited to the illustrations described and shown herein, which are deemed to be merely illustrative of the best modes of carrying out the invention, and which are susceptible of modification of form, size, arrangement of parts and details of operation. The invention rather is intended to encompass all such modifications which are within its spirit and scope as defined by the claims.

What is claimed is:

1. A method for reducing side effects while treating a mammal using indole-3-carbinol, comprising administering an effective amount of indole-3-carbinol along with side effect-reducing amounts of a side-effect reducing component.

2. The method of claim 1, wherein said side effect-reducing component includes at least one agent selected from the group consisting of vitamin C, selenium, folic acid, pyridoxine (B6), cyanocobalamin (B12) and combinations thereof.

3. The method of claim 2, wherein said side effect-reducing component comprises vitamin C.

4. The method of claim 3, wherein said indole-3-carbinol is administered in an amount of at least about 100 mg/day/100 lbs.

5. The method of claim 3, wherein said indole-3-carbinol is administered in an amount between about 150 and about 1000 mg/day/100 lbs.

6. The method of claim 3, wherein said indole-3-carbinol is administered in an amount between about 200 and 500 mg/day/100 lbs., and said vitamin C is administered in amounts between about 500 and about 8000 mg/day/100 lbs.

7. The method of claim 2, wherein said side effect-reducing component comprises vitamin C, selenium, folic acid, pyridoxine (B6) and cyanocobalamin (B12).

8. The method of claim 2, wherein said indole-3-carbinal is administered in amounts between about 200 and 500 mg/day/100 lbs., and said side effect-reducing component comprises:
vitamin C—500-8,000 mg/day/100 lbs.
selenium—70-700 mcg/day/100 lbs.
folic acid—400-2,000 mcg/day/100 lbs.
pyridoxine (B6)—2-40 mg/day/100 lbs.; and
cyanocobalamin (B12)—6-10 mcg/day/100 lbs.

9. The method of claim 2, wherein said side effect-reducing component further comprises at least one additional agent selected from the group consisting of vitamin A, vitamin D, vitamin E, vitamin K, thiamin (B1), Niacinamide (B3), Biotin, pantothemic acid, magnesium, zinc, copper, chromium, lycopene, catechin, resveratrol and combinations thereof.

10. The method of claim 1, wherein the mammal is a human.

11. The method of claim 1, wherein the indole-3-carbinal and side effect-reducing component are administered orally.

12. The method of claim 1, wherein said indole-3-carbinal is selected from the group consisting of dimer 3,3′-dindolylmethane (DIM), indole[3,2-b]carbazole (ICZ) and combinations thereof.

13. The method of claim 1, wherein said side-effect-reducing component and said indole-3-carbinol are present in a ratio by weight of said side-effect-reducing component to said indole-3-carbinol of at least about ¼.

14. The method of claim 13, wherein said ratio by weight is at least about 1/1.

15. The method of claim 13, wherein said ratio by weight is at least about 10/1.

16. An indole-3-carbinol-containing composition comprising an effective amount of indole-3-carbinol and a side effect-reducing amount of a side-effect reducing component.

17. The composition of claim 16, wherein said side effect-reducing component includes at least one agent selected from the group consisting of vitamin C, selenium, folic acid, pyridoxine (B6), cyanocobalamin (B12) and combinations thereof.

18. The composition of claim 16, wherein said side effect-reducing component comprises vitamin C.

19. The composition of claim 16, wherein said composition includes indole-3-carbinol in an amount of at least about 100 mg/dose.

20. the composition of claim 16, wherein the composition includes indole-3-carbinol in amount between 150 and about 1000 mg/dose.

21. The composition of claim 16, wherein said composition includes indole-3-carbinol in an amounts between about 200 and 500 mg/dose, and vitamin C in an amount between about 500 and about 8000 mg/dose.

22. The composition of claim 16, wherein said side effect-reducing component comprises vitamin C, selenium, folic acid, pyridoxine (B6) and cyanocobalamin (B12).

23. The composition of claim 16, wherein said composition includes indole-3-carbinal in an amount between about 200 and 500 mg, and said side effect-reducing component comprises:
vitamin C in an amount between 500 mg/dose and about 8,000 mg/dose;
selenium—70-700 mcg/dose;
folic acid—400-2,000 mcg/dose;
pyridoxine (B6)—2-100 mg/dose; and
cyanocobalamin (B12)—6-100 mcg/dose.

24. The composition of claim 16, wherein said side effect-reducing component further comprises at least one additional agent selected from the group consisting of vitamin A, vitamin D, vitamin E, vitamin K, thiamin (B1), Niacinamide (B3), Biotin, pantothenic acid, magnesium, zinc, copper, chromium, lycopene, catechin, resveratrol and combinations thereof.

25. The composition of claim 16, wherein said indole-3-carbinal is selected from the group consisting of dimer 3,3′-dindolylmethane (DIM), indole [3,2-b]carbazole (ICZ) and combinations thereof.
